(19) United States
(12) Patent Application Publication
(10) Pub. No.: US 2012/0073358 A1
(43) Pub. Date: Mar. 29, 2012

(54) ELECTRODEPOSITED GOLD NANOSTRUCTURES

(75) Inventors: Suresh Bhargava, Melbourne (AU); Samuel James Ippolito, Melbourne (AU); Ylias Mohammad Sabri, Melbourne (AU)

(73) Assignee: RMIT Unversity, Melbourne (AU)

(21) Appl. No.: 13/375,373

(22) PCT Filed: May 31, 2010
(86) PCT No.: PCT/AU2010/000662

§ 371 Date: Nov. 30, 2011

(30) Foreign Application Priority Data
Jun. 1, 2009 (AU) 2009902459

(51) Int. Cl. G01N 33/20; B32B 5/16; B32B 15/01; C25D 5/00; C25D 3/48

(52) U.S. Cl. 73/31.05; 205/112; 205/96; 428/553

(57) ABSTRACT

A mercury vapour sensor in which the sensor surface is a gold substrate, and gold nanostructures with controlled crystallographic facets are strongly adhered to the substrate. A substantial increase in response magnitude and stability of a quartz crystal microbalance (QCM) based mercury vapour sensor is achieved using this sensor surface. The method of forming gold nanostructures on a gold substrate includes the steps of electrodepositing gold onto a gold working electrode from a solution of hydrogen or alkali metal tetrahaloaureate (III) and an additive such as lead acetate at an electro-deposition temperature between 20 and 40° C. and a deposition time of at least 15 seconds. The growth is controlled by the composition of the deposition solution, the temperature and the current density. The deposition rates may be varied as will the deposition times which are preferably about 150 seconds but may be as long as 15 minutes. The preferred deposition solution contains 2.718 g/l of hydrogen tetrachloroaurate(III) hydrate with 0.1 to 0.5 g/l of lead acetate.

This invention relates to gold nanostructures on a metallised substrate and to methods of forming the structures by electrodeposition. The nanostructures have utility as surfaces for chemical and biological surfaces in sensors.

### BACKGROUND TO THE INVENTION

Controlling the shape of nanocrystals is one of the major goals in nanomaterials research, as shape-controlled nanocrystals have many prospects that are likely to impact upon the fields of catalysis, self-assembly, and nanodevices. A significant amount of literature is available on the syntheses of metallic nanoparticles dispersed in solutions, however very little research has been done concerning the formation of non-mobile nanostructures formed on rigid substrates.

The concept of electrodepositing various metal nanostructures to increase the surface-to-volume ratio or the surface porosity of metallic thin films has been widely investigated. The study of surface properties, together with methods for modifying them in a controlled manner has been a major topic of recent scientific research. The physico-chemical properties of nanocrystals are determined not only by the large proportion of surface atoms but also by their crystallographic structures. The former is determined by the size of the particle or nanostructure, and the latter is predominantly shape-dependent. A significant amount of research has reported the effects of size and different crystallographic planes on physico-chemical and electrical properties of nanomaterials. Such structural properties have been studied for their unique catalysis and sensing capabilities at the different crystal faces. However, the majority of distinctive capabilities of various crystallographic planes have so far been studied for nanoparticles formed in solutions.

A significant problem with many metallic nanomaterials is that they are formed in solution as suspended nanoparticles and are loosely fixed to the surface of a substrate (as is in the case of the dendritic nanostructures). This limits the applicability of metal nanoparticles for real-world applications, since assembly of rigidly adhered nanoparticles on rigid substrates is still a major challenge. Hence, a method of creating metallic nanostructures with well-defined shape, crystallographic properties and good mechanical adherence to the substrate is of the upmost importance for sensors, catalysts and a variety of other applications requiring well formed nanostructural surfaces with highly ordered interstitial spacing. The electrodeposition of gold nanostructured surfaces from gold cyanide, citrate and phosphate solutions using rotating disc electrodes has been reported. H. Y. Cheh, and R. Sard, Electrochemical And Structural Aspects Of Gold Electrodeposition From Dilute Solutions By Direct Current. Journal of the Electrochemical Society, 1971. 118(11): p. 1737-&.

However, there have only been isolated attempts for shape-controlled synthesis of nanomaterials on rigid surfaces. Electrochemical methods can play a key role in achieving this goal, since these methods have the potential to incorporate metal ions into nanostructures with a range of well-defined morphologies in bulk quantities. For example, anodization processes have been used for the formation of nanoporous films of TiO₂ on silicon substrates. Likewise, the utilisation of nanochannel alumina foil templates to form arrays of Au nanotubes have been synthesised by electrodeposition.

Recent developments in the electrodeposition of Ni and Ni-based alloys, Cu and Ag have further rejuvenated interest in conformal and nanoporous coatings, as well as nanostructural deposition by electrodeposition techniques. Electrodeposited bimetallic Au/Pt nanoflowers and dendritic nanostructures of Ag have just recently been proposed for use in applications such as chemical sensing.

Airborne mercury (Hg) vapour released into the atmosphere can travel long distances from the originating source, thus it is considered a global environmental issue. Human exposure to mercury vapour is harmful to the brain, heart, kidneys, lungs, and immune system in people of all ages. It is important therefore to monitor Hg levels of industrial gaseous effluent streams, especially in stationary emission sources such as coal power plants and alumina refineries.

The most widely accepted method for measuring mercury in alumina refineries and coal fired power plants involves trapping the mercury in a train of impinger solutions (i.e. trapping the mercury vapour in liquid by bubbling a fixed quantity of gas into a vessel). Thereafter, subsequent analysis of these solutions using a technique such as cold vapour atomic absorption spectroscopy (CVAAS) can be made. This method is sometimes referred to as the Ontario Hydro (OH) method. The most significant shortfall of this method is that it does not allow timely measurements to be made as the analysis is generally performed by highly trained people in an off-site laboratory.

To overcome this shortfall research and development has recently been undertaken to produce continuous mercury emission monitors (CMEMs) capable of measuring mercury primarily for the coal fired power station industry. To date no commercially available or US EPA approved CMEM has been produced for alumina refineries. The developed CMEM systems that have been described in the open literature are essentially automated (dry) versions of the OH method and involve a process for pre-treating the gas stream before it is passed to an on-line analyser. There are several technologies used in commercially available systems for mercury sensing. Some of these technologies are:
Cold Vapour Atomic Absorption Spectrometry (CVAAS)
Atomic Fluorescence Spectrometry (AFS)
UV Differential Optical Absorption Spectroscopy
Inductively Coupled Plasma—Atomic Emission Spectrometry (ICP-AES)
Resistive Gold Film Sensor (RGFS)

The underlying mechanism for CVAAS, AFS, ICP-AES work on the absorption and emission of 253.7 nm wavelength band—at which mercury is excited. Unfortunately other chemicals found in some industry streams are also excited at this wavelength, which results in inaccurate mercury readings. UV Differential Optical Absorption Spectroscopy would suffer from similar issues as it works on similar principles. This invention is particularly concerned with developing a gold sensor surface for the detection of mercury vapour in industrial effluent streams where interference from volatile organic compounds (VOCs), water vapour and ammonia is common. Electrodeposited gold and porous gold has been shown to improve the sensitivity of a Quartz Crystal Microbalance (QCM) for Biosensing. Mostly this type of surface relies on the increased surface to volume ratio achieved by the electrodeposition process. U.S. Pat. No. 5,992,215 discloses a sensor using a copper or gold coated crystal surface in which the sensitivity is increased by using a dual delay line surface acoustic wave (SAW) sensor to cancel out extraneous environmental effects. The device also includes a heater.

It is an object of this invention to provide an improved gold nanostructured surface that is useful as a robust mercury vapour sensor element which is suitable for both industrial flue gas applications as well as small hand held units.

### BRIEF DESCRIPTION OF THE INVENTION

To this end the present invention provides a method of forming gold nanostructures on either a metallic or carbon substrate which includes the steps of electrodepositing gold onto a metallised working electrode from a solution of hydrogen or alkali metal tetrahaloaureate (III) and a growth directional additive, at an electrodeposition temperature between 20 and 40° C. and a deposition time of at least 15 seconds. This method produces a gold nanostructured surface having shaped gold nanostructures projecting from the substrate to which the nanostructures are strongly adhered. The substrate may be any suitable metal such as copper but is preferably gold. The preferred gold compound is hydrogen tetrachloroaurate(III) hydrate with lead (IV) acetate. The lead compound may be substituted with other directional controlling compounds such as various lead (II) salts, halides, saccharin, Nafion, CTAB, SDS, Triton, and cysteine.

Nafion is a sulfonated tetrafluoroethylene based fluoropolymer-copolymer preferably Nafion-117, which is perfluorosulfonic acid-PTFE copolymer Triton is a Polyethylene glycol octylphenol ether

For example: Triton X-114 is Poly(oxy-1,2-ethanediyl),a[(1,1,3,3-tetramethylbutyl)phenyl]-w-h, Chemical Formula: C₈H₁₆C₆H₄(—CH₂CH₂O)10H CTAB is Cetyl trimethylammonium bromide (C₁₆H₃₃)N(CH₃)₃Br SDS is Sodium Dodecyl Sulfate (C₁₂H₂₅NaO₄S)

Morphology is just as important as crystalline structure for different applications. The SEMS data (which detail morphology) and the XRDs (which detail crystallinity) described in the examples below indicate that in this invention the method controls both by slight changes in the deposition conditions can be used to tailor both parameters.

Controlling the shape of nanocrystals is one of the major goals in nanomaterials research, as shape-controlled nanocrystals have many prospects that are likely to impact upon the fields of catalysis, self-assembly, and nanodevices. A significant amount of literature is available on the syntheses of metallic nanoparticles dispersed in solutions, however very little research has been done concerning the formation of non-mobile nanostructures formed on rigid substrates. In this invention metallic nano-structured surfaces are formed on rigid substrates. Special emphasis is placed on size, shape and preferential crystallographic growth of these metallic nanostructures. The growth is controlled by the composition of the deposition solution, the temperature and the current density. The deposition rates may be varied as will the deposition times, which are preferably about 150 seconds, but may be as short as 90 seconds or as long as 15 minutes, depending on whether a two or three electrode system is employed or what the chosen current density of the deposition protocol uses. The preferred deposition solution contains 2.718 g/l of hydrogen tetrachloroaurate(III) hydrate with 0.1 to 0.5 g/l of lead acetate. It should be noted that by using higher concentrations of up to 9 g/l of hydrogen tetrachloroaurate(III) hydrate will result in the formation of thick nanospike structures.

In this invention these structures are used for the sensing of mercury vapour in the presence of volatile organic compounds (VOCs) found in industrial effluent streams.

This invention shows that highly oriented and ornate gold nanostructures with controlled crystallographic facets substantially increase the response magnitude and performance of a QCM based mercury vapour sensor over operating periods spanning several consecutive months. Additionally the sensor surface is able to work well in the presence of interfering volatile organic compounds (VOCs) that are found in many industrial effluent streams.

In another aspect of this invention there is provided a mercury vapour sensor in which the sensor surface is a gold substrate to which gold nanostructures with controlled crystallographic facets are strongly adhered to the substrate.

The sensor of this invention uses well established technology known as Quartz Crystal Microbalances (QCMs). QCMs are part of a wider family of single element sensors based on Thickness Shear Mode (TSM) acoustic resonators (which are also called Bulk Acoustic Wave (BAW) devices). They have no moving parts and work by measuring very small mass changes (4.24 ng/cm².Hz) at the surface of the sensor using the acoustic-electric phenomenon. Since mercury is a heavy element, it is atomically much heavier than other gases and organic vapours present in an alumina refinery stream. Therefore as the mercury molecules interact with the surface of the QCM based sensor, the Hg atoms register a higher mass (weight) on the surface comparative to other interactions. This interaction is facilitated by using a sensitive layer formed from gold which has high affinity towards Hg atoms. In preferred aspects of this invention the gold sensitive layers can have more than 3 times larger surface area than evaporated gold surfaces and have superior selectivity towards mercury interactions in the presence of interfering gases.

In the context of alumina refineries, trace quantities of Hg have been found in emissions from various sources, in particular: oxalate kiln, digestion, calciners, and other minor sources such as liquor burner and boilers within the Bayer process—the Bayer process is the name given to the chemical processes used in alumina refineries. Depending on the origin of the (bauxite) ore, mercury contents between 50 mg and 431 mg per tonne of bauxite have been reported. During the refinery process much effort is made to capture the mercury before it is emitted into the environment, however measurable quantities of Hg are still emitted for every metric tonne of alumina produced. An estimate of approximately 2.9 tonnes of mercury is vapour was emitted by Australian alumina refineries in a one year period spanning 2006-2007.

In order to better understand mercury emission sources, migration, and environmental and societal impacts of Hg vapour, continuous mercury emissions monitors (CMEMs) located at strategic points within the Bayer process are imperative. For example, the sensor could be located at the digestion or evaporation stacks, or at the output of a Regenerative Thermal Oxidizer (RTO) to allow operators to determine the primary process where mercury is most likely to escape in the gas phase.

Using the surface of this invention a substantial increase in response magnitude and stability of a quartz crystal microbalance (QCM) based mercury vapour sensor has been achieved via a developed surface modification technique employing an electrochemical route. Using this technique, strongly adhered and well formed nanostructures are grown to the surface of the gold electrode of the QCM in a uniform and controlled fashion. The QCM based sensor deals well with a range of interfering gases (such as: Ammonia, Sulphur dioxide, Acetone, Dimethyl disulphide, Ethyl Mercaptan, Methyl Ethyl Keytone, Acetaldehyde, etc.) and has the potential to overcome other interfering volatile organic compounds (VOCs) that are found in many industrial effluent streams such as Alumina refineries and coal power stations streams. It should be noted that the developed surface, although applied to a QCM in the context of this project, would equally be able to be applied to other platforms that work on either conductometric (chemiresistive) or mass based sensing mechanisms. For example, the family of Surface Acoustic Wave (SAW) devices would be most suitable for low Hg concentration measurements in the parts per billion range. Well-formed nano-engineered surfaces have great potential for many applications, such as: ultrasensitive layers in chemical- and bio-sensing; for enhanced catalytic efficiency; Surface Enhanced Raman Spectroscopy (SERS) substrates, self cleaning surfaces; and in fuel cell technology. It should also be noted that Au is a biocompatible material and the high surface-to-volume ratio of the electrodeposited structures would be most suitable for many bio-sensing applications.

Additionally the highly ordered interstitial spacing of the nanospikes would also have similar or better super-hydrophobic properties than those observed for pyramidal structures. The surfaces of this invention exhibit a good degree of interstitial spacing which will lead to the formation of an air-bilayer between a droplet and the surface, which is the basis of the lotus leaf effect displayed in natural superhydrophobic surfaces. By controlling the electrodeposition parameters, it is possible to form hierarchical nanostructures with two-tier roughness in the form of secondary nodes on the primary structures, thus further increasing the superhydrophobicity of these surfaces. Similarly, the secondary nodes would also further enhance the sensing and catalytic abilities, by increased defect sites and surface-to-volume ratio.

### DETAILED DESCRIPTION OF THE INVENTION

Preferred aspects of the invention will be described with reference to the drawings in which:

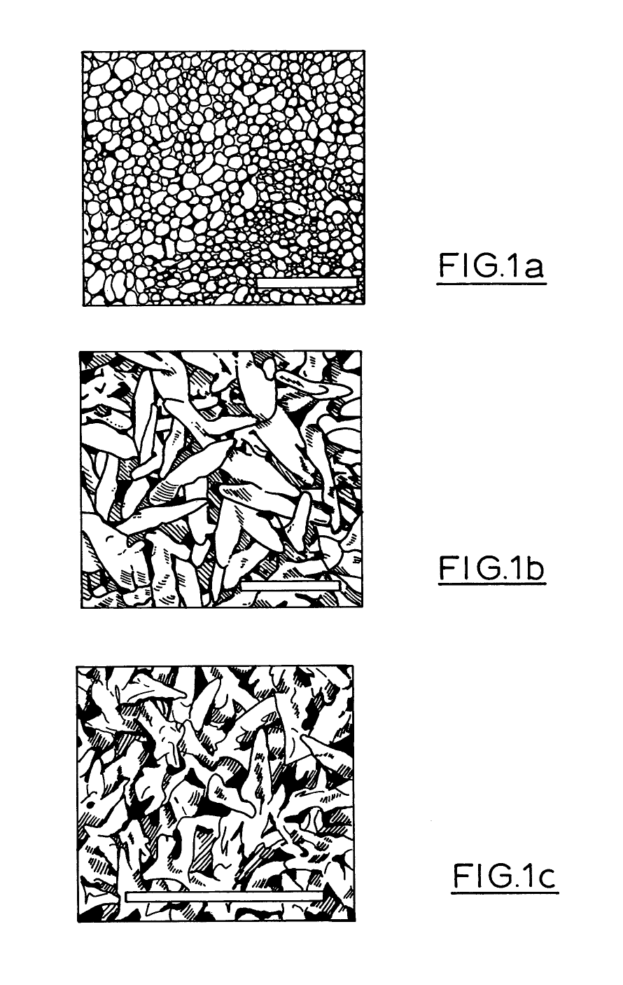

FIG. 1 a) shows a Scanning Electron Microscope (SEM) image of a non-modified gold electrode surface (prior art) and b) an SEM image of a preferred surface of this invention and c) larger and thick nanospike structured formed using higher concentrations of hydrogen tetrachloroaurate(III) hydrate electrolyte solution;

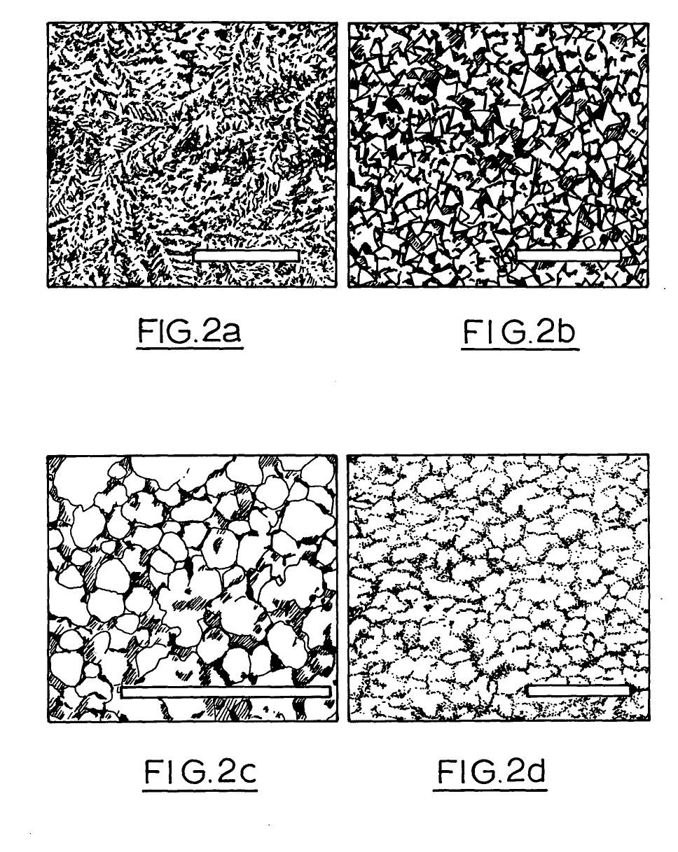

FIG. 2 shows an SEM image of a) a nanodendrite gold surface (prior art) and b) through to d) are some alternative nanostructured surfaces of this invention;

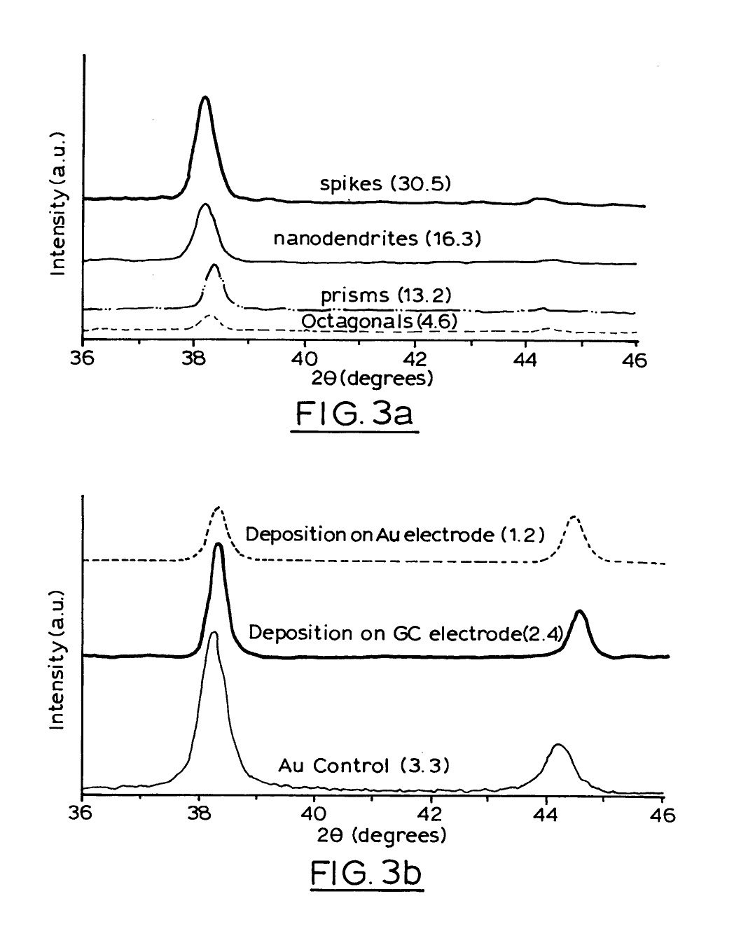

FIG. 3 illustrates the GADDS patterns of the different electrodeposited structures, where a) shows that of FIG. 1 b) and FIGS. 2 a) b) and c), and FIG. 3 b) shows that of FIG. 1 a) and FIG. 2 d);

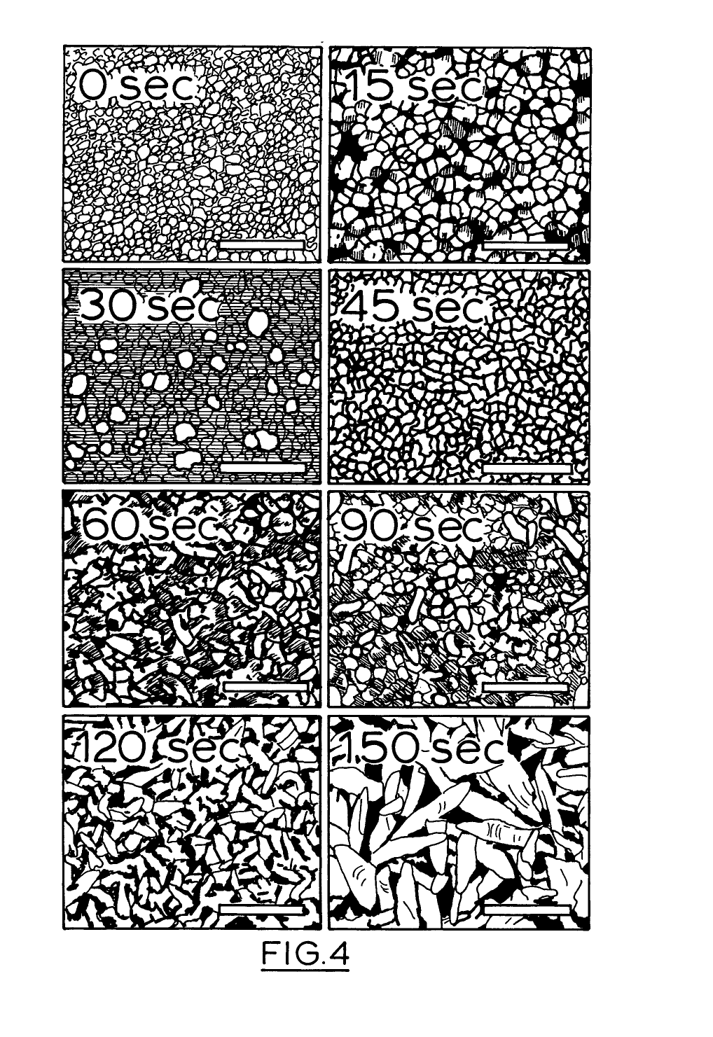

FIG. 4 shows SEM images of nanostructures with increasing deposition times;

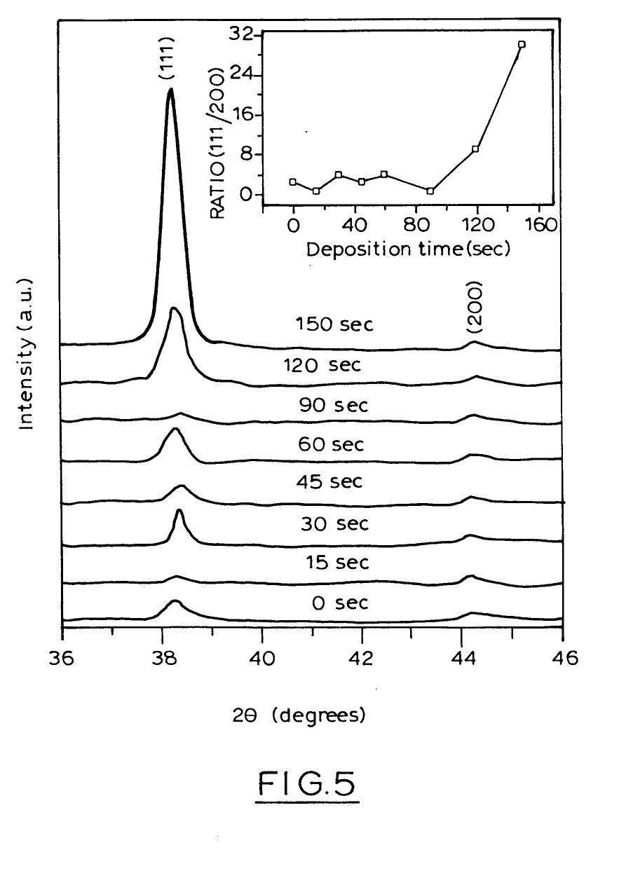

FIG. 5 illustrates the GADDS pattern of the structures shown in FIG. 4;

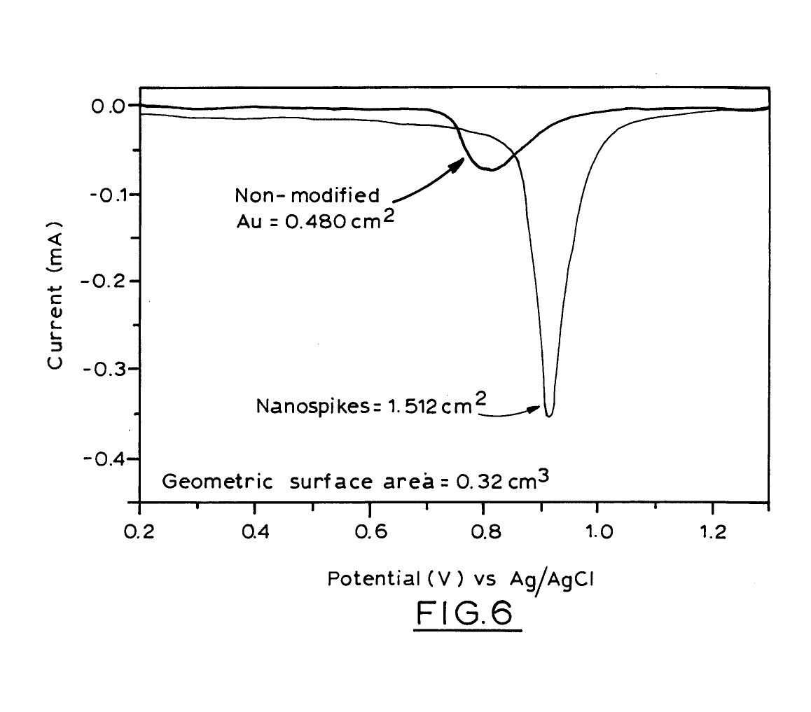

FIG. 6 shows the electrochemical surface measurements of the surfaces shown in FIGS. 1 a) and b);

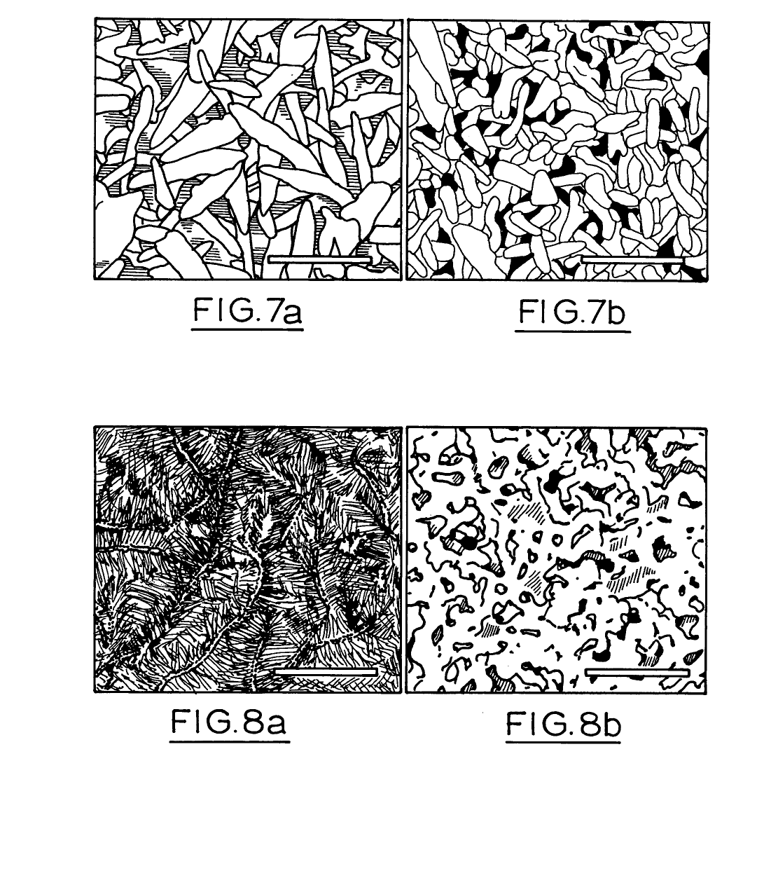

FIG. 7 shows SEM images of the structure of FIG. 1 b) before and after heat treatment;

FIG. 8 shows SEM images of the structure of FIG. 2 a) before and after heat treatment;

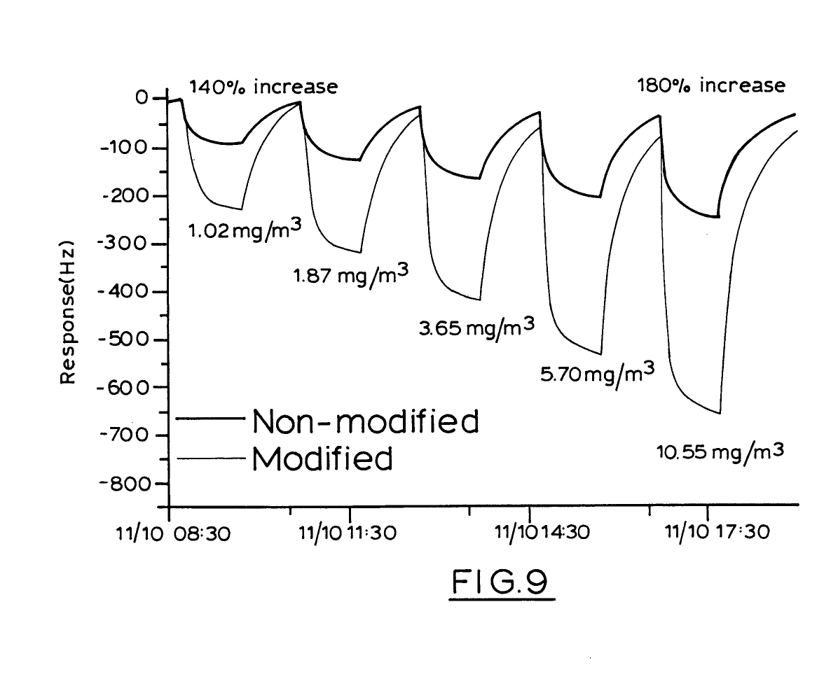

FIG. 9 illustrates comparative sensor response of non-modified and Nanospike QCM sensors towards mercury vapour when operating at 89° C.;

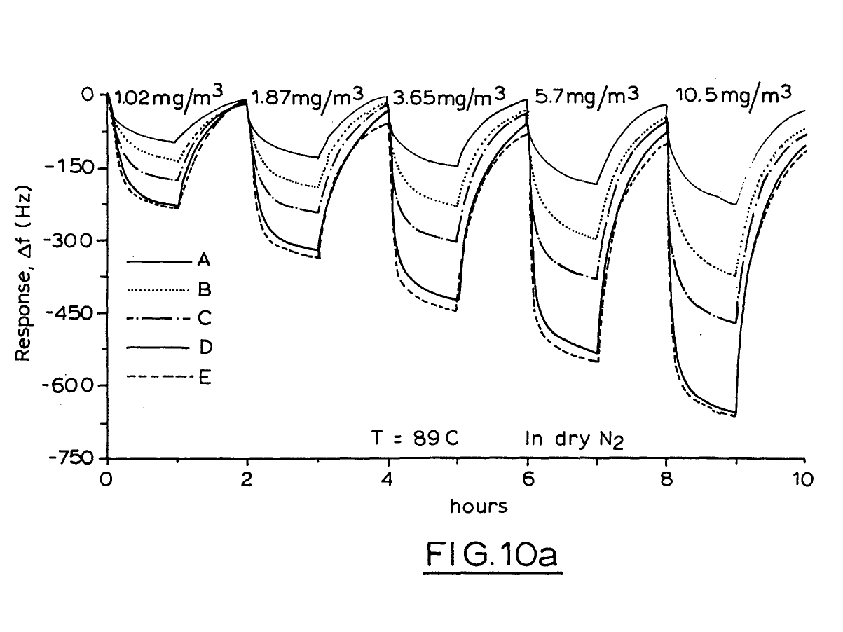

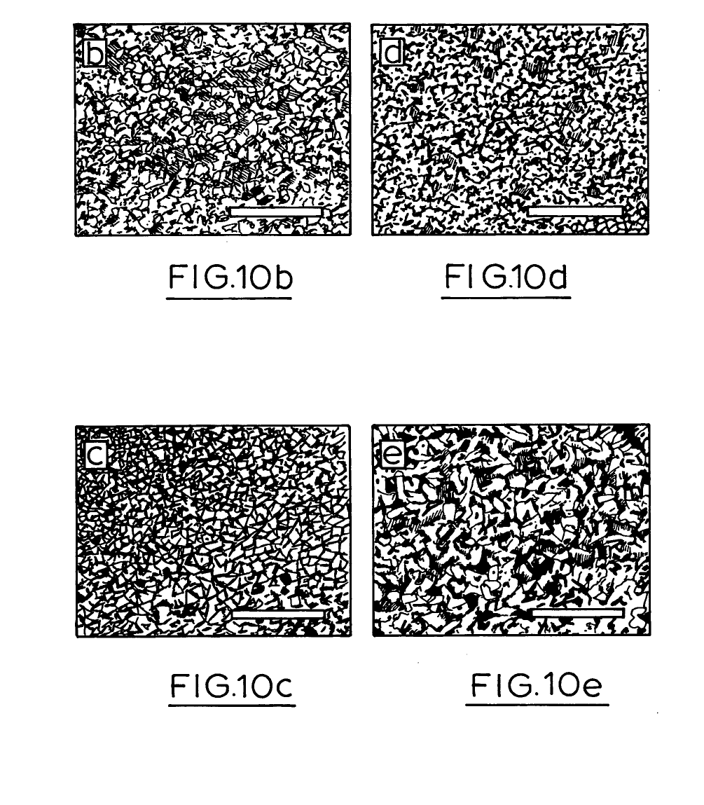

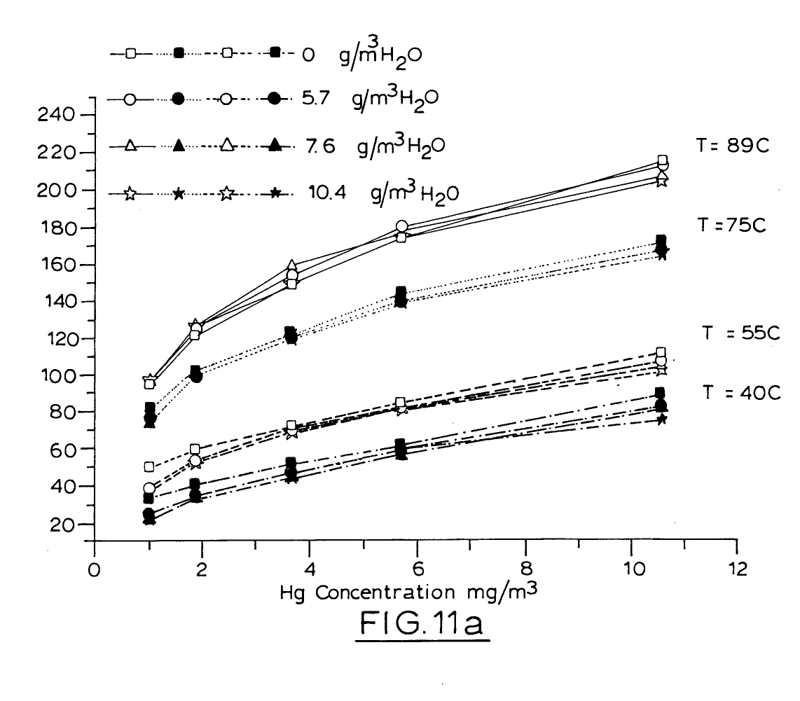

FIG. 10 illustrates comparative sensor responses and corresponding SEM images of a range of electrodeposited surface;

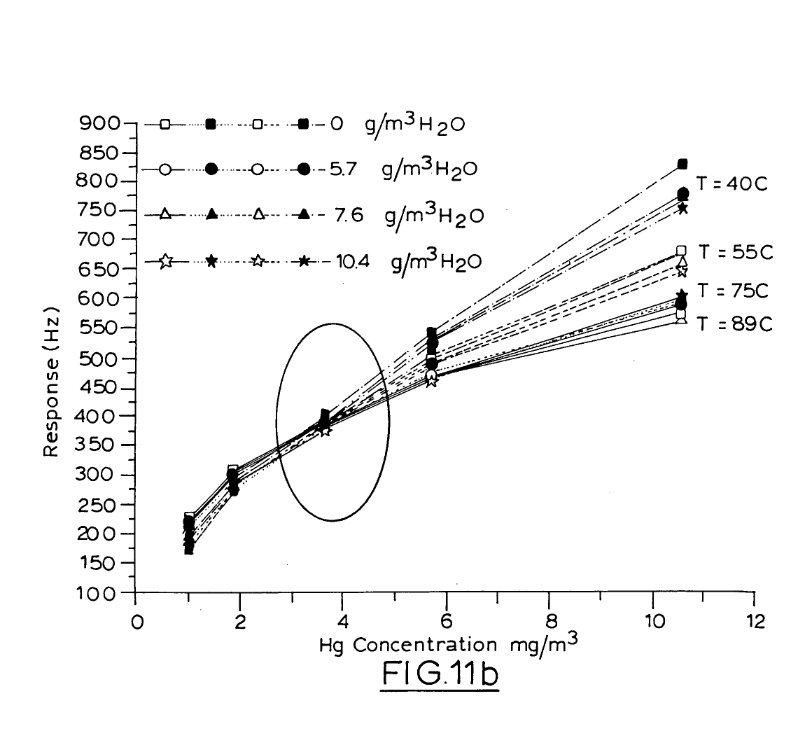

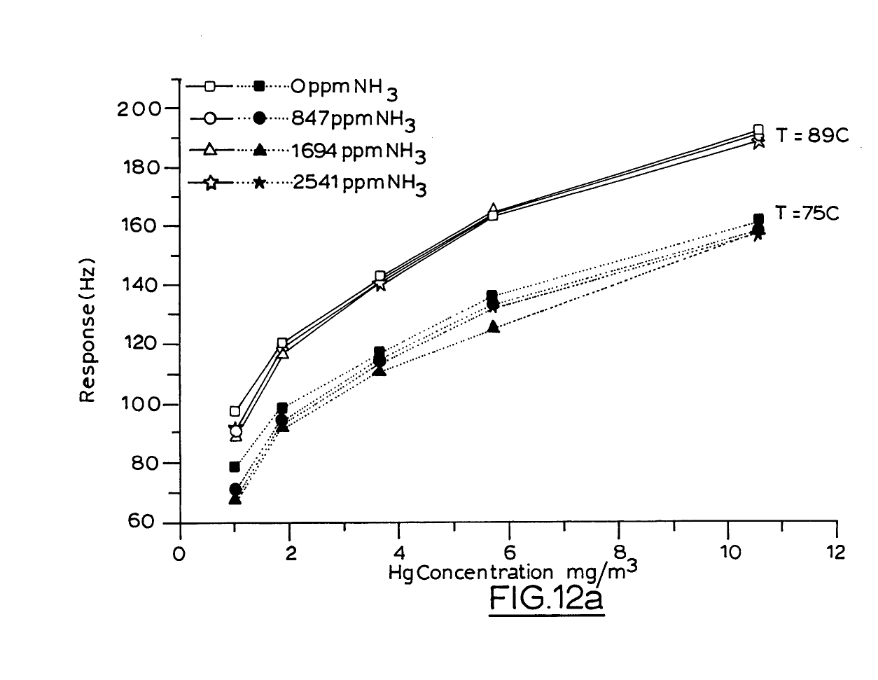

FIG. 11 shows comparative response of non-modified and Nanospike QCM sensor in the presence of different levels of (low) humidity interference and operating temperatures, when prepared according to this invention;

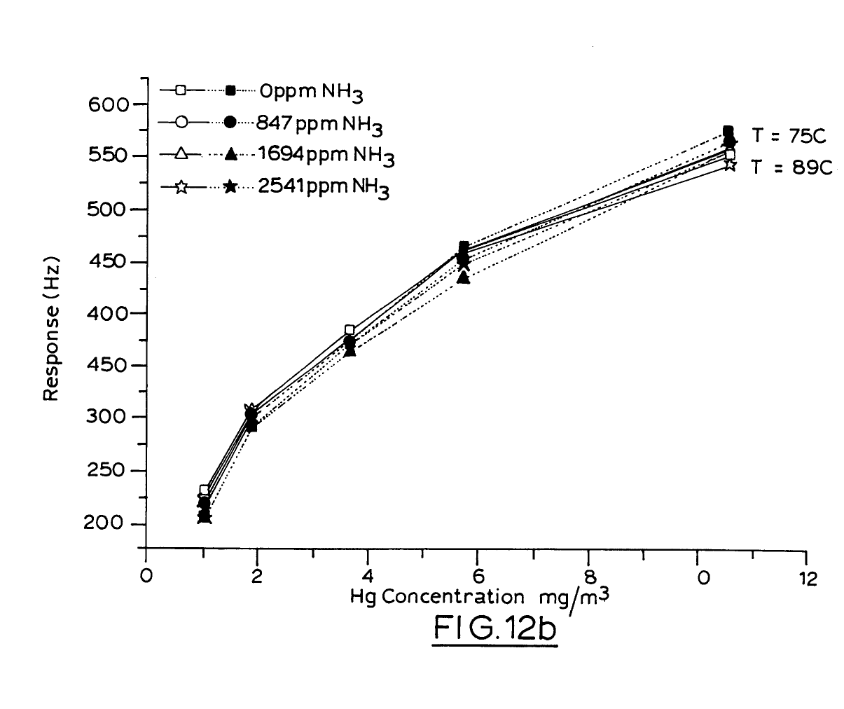

FIG. 12 shows a comparative effect of ammonia interference and operating temperature on sensor response;

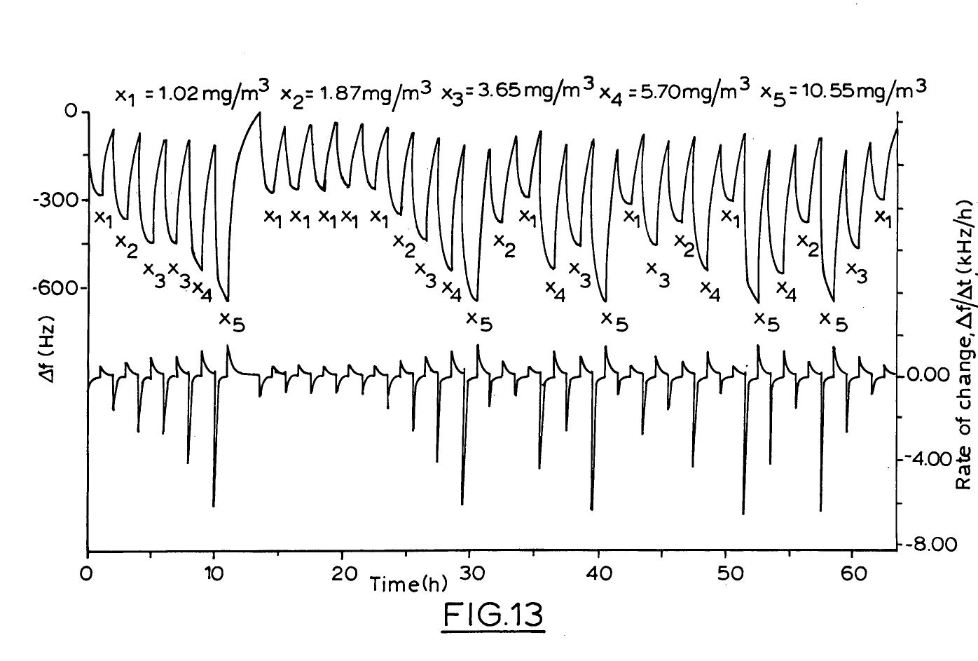

FIG. 13 shows factorial test patterns for 5 concentrations of mercury at an operating temperature of 89° C. (both Δf and rate of change Δf/Δt are shown);

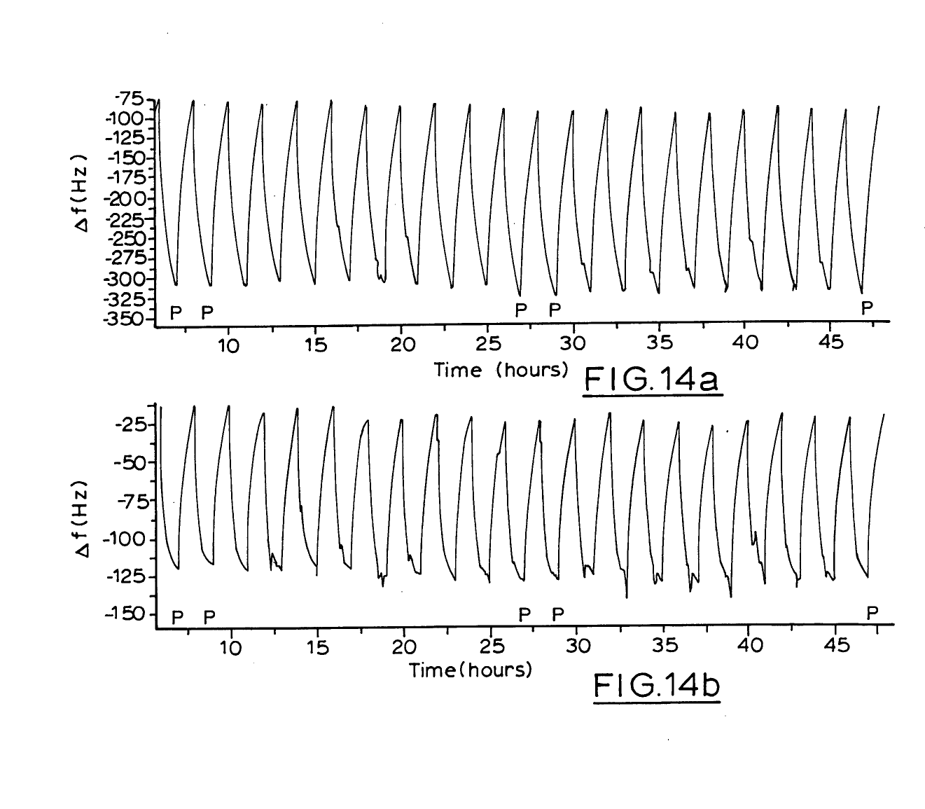

FIG. 14 shows continuous pulses of mercury (3.65 mg/m³) in the presence of interfering gas species such as Ammonia, Dimethyl disulphide, Ethyl Mercaptan, Methyl Ethyl Keytone, Acetaldehyde and high levels of water vapour;

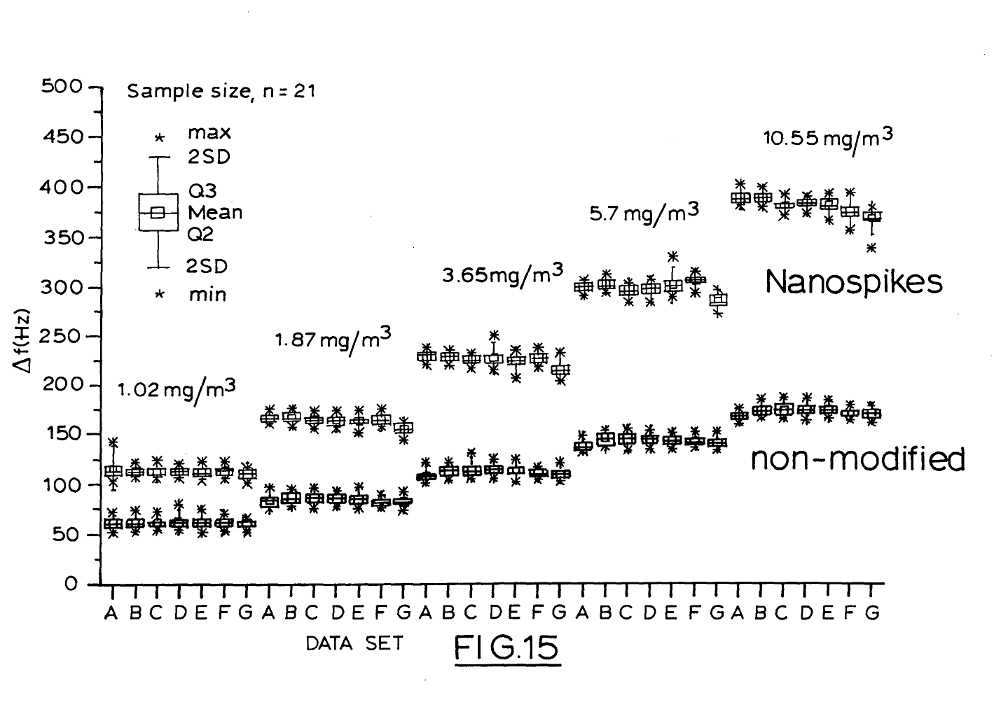

FIG. 15 shows the performance summary for the adsorption phase for the non-modified and electrodeposited (nanospike) sensors at an operating temperature of 102° C. in the presence of interfering gas species—Data was acquired over 4 months of continuous testing period by repeating the testing sequence shown in FIG. 14 seven times for each of the 5 tested mercury vapour concentration;

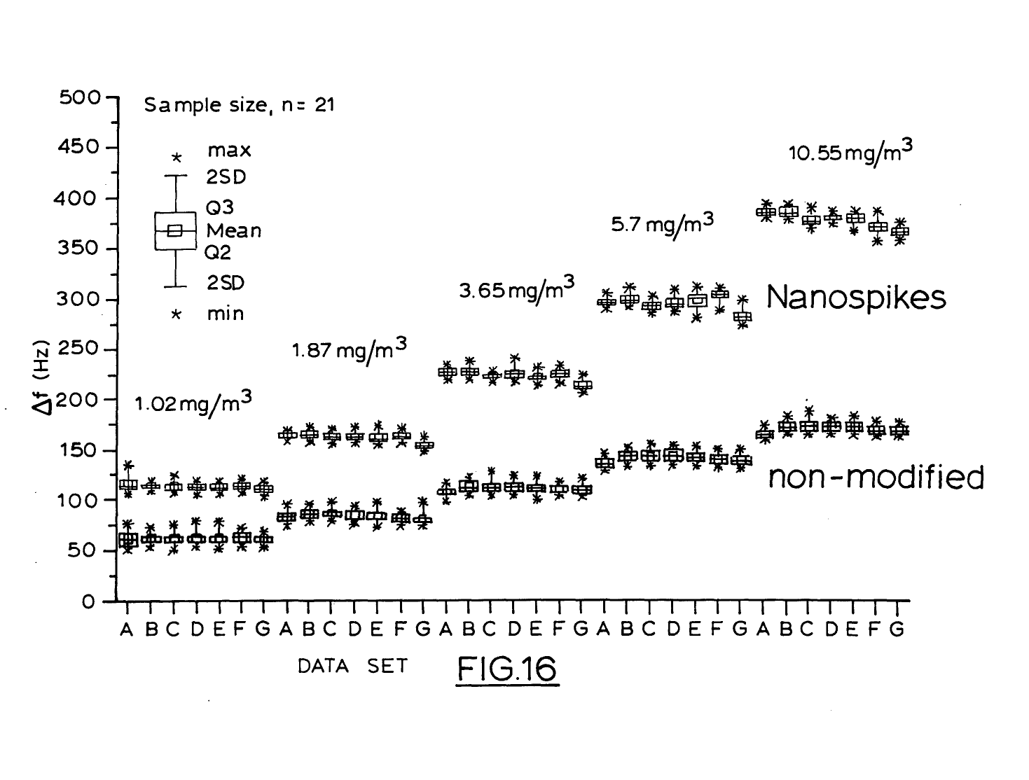

FIG. 16 shows the performance summary for the desorption phase for the non-modified and electrodeposited (nanospike) sensors over 4 months of continuous testing at an operating temperature of 102° C. in the presence of interfering gas species;

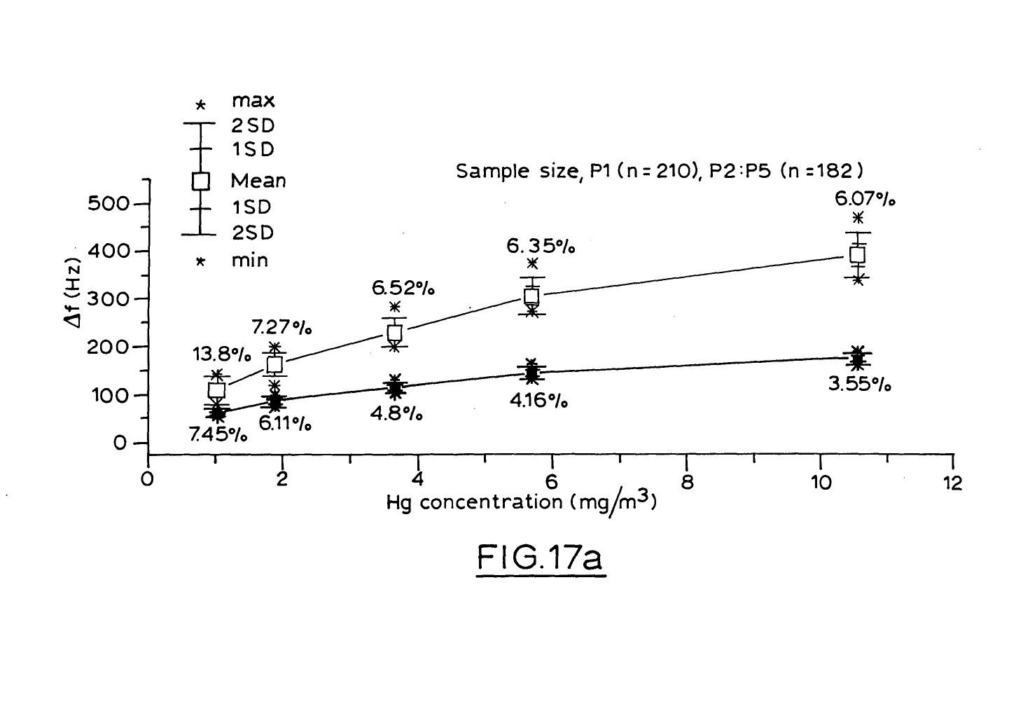

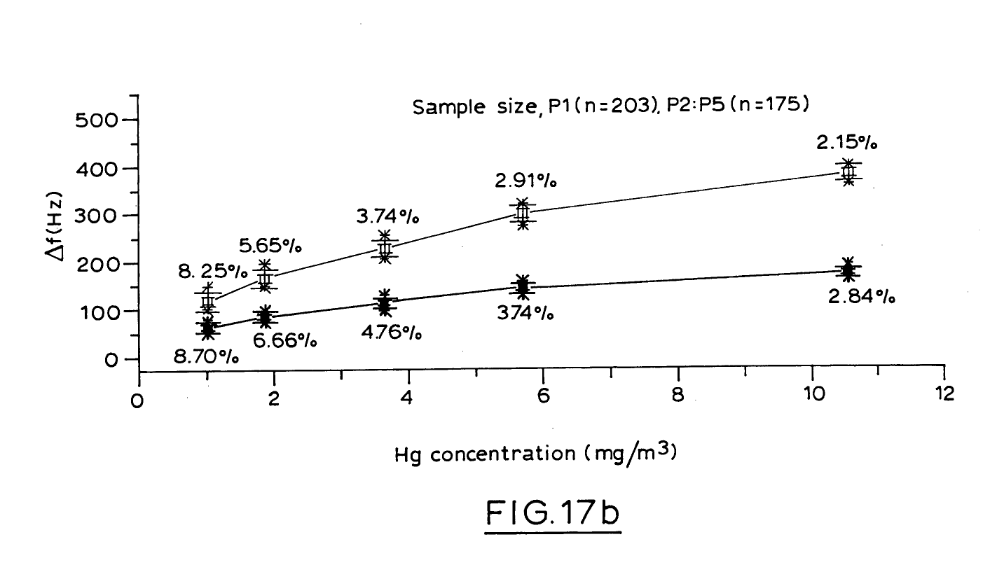

FIG. 17 is a summary of the comparison between the non-modified sensor and the sensor of this invention over the 4 month testing period—the calculated coefficient of Variance (CoV) value is shown for each data point in the calibration curve;

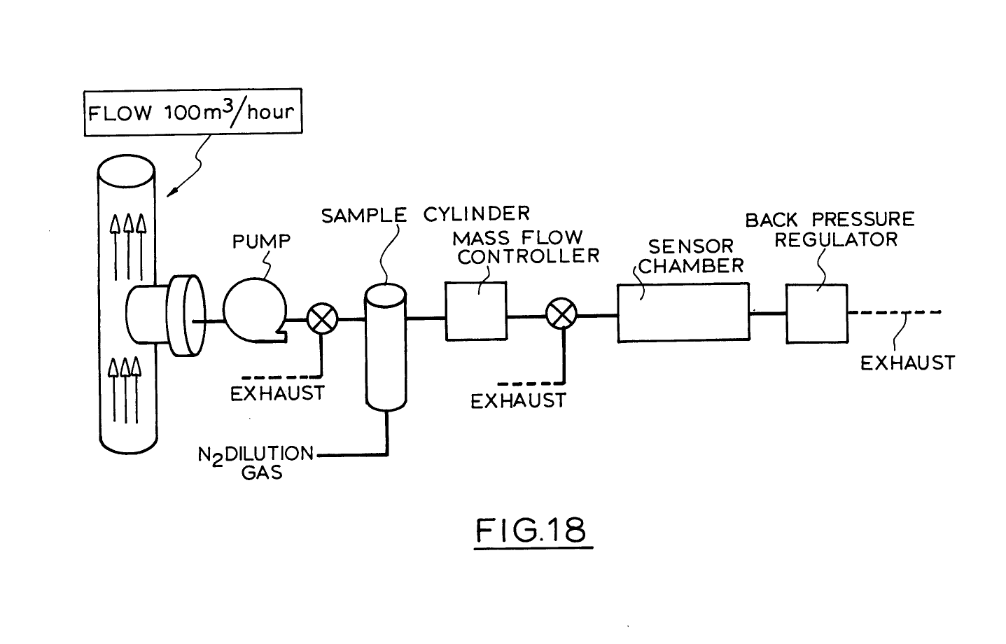

FIG. 18 illustrates the sensor arrangement of this invention using an extractive dilution technique.

A preferred deposition method of this invention will be described with reference to the application of the gold nanostructured surface as a sensing surface for a Quartz Crystal Microbalance (QCM).

In this example the plating solution contained 2.718 g/l hydrogen tetrachloroaurate (III) trihydrate and 0.177 g/l lead (II) acetate. The concentration of the hydrogen tetrachloroaurate (III) trihydrate and lead acetate can range as high as 9 g/l and 0.5 g/l, respectively, to give alternative nanostructures. The preferred parameters to achieving the nanostructures of interest are:

a) In a two electrode system:
electrolyte with 2.718 g/L hydrogen tetrachloroaurate (III) trihydrate and 0.177 g/L lead (II) acetate. In this case a deposition solution volume 10 to 75 ml was used.
Electrodeposition temperature between 20 and 25° C.
Inert or gold counter electrode.
Gold coated QCM as the working electrode; both sides of the QCM were used simultaneously.
Chloride ion concentration was maintained in excess at about 30 mM.
Deposition times between 15 seconds to 150 seconds.
The QCM was in a stationary position during the electrodeposition process.
A spacing of 2.5 cm was maintained between the anode and cathode.
The electrolyte was stirred in a constant fashion using a magnetic stirrer.
Operational modes can vary and may be based on
   Constant current: Current between 0.1 mA and 5 mA (depending on exposed Electrode surfaces—we used a total area of 0.32 cm² over both electrodes that form the QCM).
   Constant voltage: Using a constant potential difference between 0.2V and 2V.

b) In a three electrode system:
electrolyte with 2.718 g/L hydrogen tetrachloroaurate (III) trihydrate and 0.177 g/L lead (II) acetate with a total volume of 5 to 10 ml.
Electrodeposition temperature between 20 and 25° C.
Inert counter electrode with Ag/AgCl reference electrode.
Gold coated QCM as the working electrode; both sides of the QCM were used simultaneously.
Chloride ion concentration was maintained in excess of about 30 mM.
Deposition times between 5 to 15 minutes.
The QCM was in a stationary position during the electrodeposition process.
Using a constant potential difference between 0V and 0.5V (when the pH of the solution is below 2.5).

The effect of various electrodeposition parameters, such as electrode separation distances, electrolyte concentration, deposition potential, deposition time, electrolyte temperature, etc., is known to determine the type of structures and surface morphology that is grown during the electrodeposition process. Additionally, the effect of different electrolytes with buffers (such as: acetate and citrate) as well as known additives (saccharine, CTAB, Nafion, SDS, Triton, cysteine, Pb⁺² and I⁻ ions) will also significantly effect the structures grown.

The significance of the electrodeposition method for shape-controlled synthesis of the nanospikes is shown in FIGS. 1b and 1c. These are the structures used for the long term Mercury Sensing work.

FIG. 1a

This is the non-modified e-beam deposited gold surface that we use. The surfaces shown in FIG. 1b and 1c (and for that matter all others I have given you) were formed on top of this type of surface.

FIG. 1b

This surface was deposited using the following parameters:
Used a 2 electrode system
150 second deposition time
2.718 g/l hydrogen tetrachloroaurate (III) trihydrate and 0.177 g/l lead (II) acetate solution
2V potential difference between electrodes
2.5 cm electrode separation

FIG. 1c

This surface was deposited using the following parameters:
Used a 3 electrode system
10 minute deposition time
8.1 g/l hydrogen tetrachloroaurate (III) trihydrate and 0.177 g/l lead (II) acetate solution
At a deposition potential of 0.05V when using a Ag/AgCl reference electrode

Electrode separation distance is not important when using a 3 electrode deposition system as we were using a reference electrode.

Alternative structures as shown in FIG. 2 are also possible by adjusting deposition conditions.

FIG. 2a
Used a 2 electrode system
20 second deposition time
27.18 g/l hydrogen tetrachloroaurate (III) hydrate and 1.77 g/l lead (II) acetate solution
−2V potential difference between electrodes
2.5 cm electrode separation

FIG. 2b
Used a 2 electrode system
120 second deposition time
2.718 g/l hydrogen tetrachloroaurate (III) hydrate and 0.177 g/l lead (II) acetate solution
Chloride ion concentration below 2 M.
−2V potential difference between electrodes
2.5 cm electrode separation

FIG. 2c
Used a 2 electrode system
120 and 150 second deposition time
2.718 g/l hydrogen tetrachloroaurate (III) hydrate and 0.177 g/l lead (II) acetate solution
Chloride ion concentration below 2 M.
−2V potential difference between electrodes
2 cm electrode separation

FIG. 2d
Used a 3 electrode system
10 minutes deposition time
8.7 g/l potassium tetrabromoaurate in a 1% Nafion solution
−0.35V potential difference between electrodes

The nanostrucutres shown in FIG. 2b (nanoprisms) and 2c (nano-octagonal) have also been tested for mercury vapour sensing and show comparable results to the nanospikes.

These nanospikes, nanoprisms and nanoctagonals have not previously been used for mercury sensing. These nanostructures show increase in response magnitude and sensor stability for mercury vapour sensing, and that the sensor is capable of dealing with both high levels of humidity (water vapour) and various other chemical and Volatile Organic Compounds (VOCs) interfering gas species that are found in many industrial effluent streams. These include, but are not limited to: Ammonia, Sulphur dioxide, Nitrogen dioxide, Nitrogen monoxide, Alcohols, Acetone, Dimethyl disulphide, Ethyl Mercaptan, Methyl Ethyl Keytone and Acetaldehyde.

The dendritic (nanowire-like) structures grown on gold coated quartz substrates are shown in FIG. 2a, which are similar to those reported in the prior art. These Au nanodendrites (also sometimes referred to as ‘porous gold’ or ‘black gold’) are known to be very delicate and can be easily washed off the surfaces. Additionally the nanodendrites are very typical structures, and are widely published in the literature even with other metals such as silver and platinum. In comparison the SEM images in FIG. 1b, 1c and FIGS. 2c 2b and 2d show highly oriented and ornate nanostructures with controlled crystallographic facets that can be reproduced by electrodepositing Au onto Au-coated quartz substrates (or in the case of FIG. 2d on glass carbon substrates also). These observations are also supported by the different ratios between [111]/[200] in the GADDS profile of the respective nanostructures in FIG. 3. Apart from the nanodendrites, all the other nanostructures shown are rigidly fixed to the substrate and more importantly, they exhibit different preferential crystallographic orientation. This makes them particularly notable and attractive candidates for a range of applications requiring nano-engineered surfaces with specific physico-chemical and electrical properties.

Further significance of the electrodeposition method for shape-controlled synthesis of nanospikes (FIG. 1b, 1c and 4) is evident from the GADDS results shown in FIG. 5. The nanospikes appear to be the most promising nanostructures for mercury sensing. The results presented in FIG. 4 highlights the increase of [111] to [200] peak ratios of electrodeposited Au nanostructures in a time dependent manner as shown in FIG. 5. A significant enhancement of 800% in [111] peak was observed for the 150 second electrodeposited sample when compared to the non-modified gold surface (0 second). The corresponding SEM image shown in FIG. 4 shows nanospikes with dimensions between 100-500 nm thick and more than 1500 nm long, the tips of which are well-defined tapering triangular points. A surface area comparison of the non-modified (e-beam evaporated) gold surface and that of the electrodeposited nanospikes (shown in FIG. 1a) is presented in FIG. 6. This data shows that the as deposited nanospike surface has 3.15 times the surface area of the non-modified surface. They also have strong mechanical/cohesive strength, which do not break under ultrasonication and show good adhesive strength to the substrate when tested by the common ‘masking tape’ or ‘Scotch tape’ tests. Additionally they do not break away from the surface when scraped with steel tweezers.

The GADDS data clearly show that these nanospikes are not related to routinely electrodeposited nanowire/dendrites, which would otherwise be preferentially oriented in the [110] plane. Moreover, other Au nanostructures including nanoprisms (FIG. 2b), octagonal-shaped nanorods (FIG. 2c) and oriented gold rough surfaces (FIG. 2d) could be synthesized by electrodeposition, which indicates the capability of controlling growth of firmly-adhered metallic nanostructures in either [111], [110] or [100] crystallographic planes as shown in FIG. 3. Thus, the structural properties may be tailored to target specific applications which are preferentially facet dependent.

FIG. 7 shows the thermal stability of the nanospike surface once treated in air at elevated temperatures of 220° C. for a prolonged period of time. Although the nanostructures appear to have reduced in size slightly, they still hold their shape. In comparison, the nanodendrite structures shown in FIG. 8 have changed morphology significantly when treated in the same fashion.

It is not necessary to heat treat any samples in forming the surfaces.

The ‘as deposited’ sample shown in FIG. 7a was not heat treated. It could be used for electro catalysis, SERS or hydrophobicity experiments directly after the sample was deposited. However, for use as mercury sensors they are heat treated during the ‘sensor break-in period’ to about 130 or 180° C. This is done in the presence of mercury for a period of at least 3 or 4 days before use of the sensor, at slightly high operating temperature than used during the actual sensing process. The actual sensing process is generally performed between 80 and 110° C. In the case of the data, the first test was done at 89° C. for 70 days and the sensor used in the second test was tested at 102° C. for around 4 months. Both these sensor were heat treated during the ‘sensor break-in’ period. The first sensor was broke-in at around 138° C. The second sensor was broke-in at around 178° C. but a preferred break-in temperature is 150° C.

The nanospike sensor may be used at room temperature for sensing mercury without heat treating the surface. In this case it would have a much larger response magnitude, however it probably would not cope well with the interfering gases. For low temperature mercury experiments there is no need to heat treat the nanostructures.

FIG. 7 and FIGS. 8 show what extreme temperatures will do to the surfaces. There is no need to heat any sample above 150° C. This would not really affect the surface of the nanospikes. However as can be seen from FIG. 8 the nanodendrites are destroyed.

The preferred sensor of this invention is specifically designed to target the concentrations of mercury found in alumina refineries, where the mercury vapour concentration are typically within the wide range of 0.5 to 32 mg/m³. It should be noted that unlike coal fire power plant flue gases, only elemental mercury is found in an alumina refinery. This therefore removes the requirement to use a catalyst bed that converts oxides of mercury (such as HgCl₂) into elemental Hg. Although, if required such a bed could easily be added to our sensor system.

Also, unlike coal fire power plant flue gases where mercury concentrations are low (below 0.5 mg/m³), the mercury in particular parts of the Bayer process can reach as high as 50 mg/m³. These concentrations are significantly higher than the maximum detection limit of all the sensors shown in Table 1 (as most of these sensor systems are targeted towards coal fire power stations). Therefore the variability of the mercury concentrations found in Alumina refineries would make it hard to determine the appropriate dilution ratio of a sample given that a concentration of Hg as high as 530 mg/m³ and as low as 0.5 mg/m³ could be expected during a given sensing event.

The experimental data has demonstrated that the sensor of this invention has excellent performance between the range of 1.0 to 10.5 mg/m³, which when combined with a 1 to 4 dilution is suitable for alumina refineries. We are able to sense the mercury concentration between this range when the stream is contaminated with the following interfering gas species:

Water vapour
Ammonia
Acetone
Dimethyl disulphide
Ethyl Mercaptan
Methyl Ethyl Keytone
Acetaldehyde

Due to the low molecular mass of DMDS, Ethyl Mercaptan, MEK, and Acetaldehyde only a very marginal effect on sensor response was observed. We have also exposed the sensor to SO₂ and NOₓ mixes which partially simulate the stream of a coal-fired power station. In this case the stream has upwards of 3000 times the concentration of SO₂ found in the Bayer Process (alumina refineries). The sensor was found to work under these conditions, however more work needs to be performed to determine if the sensor truly can be used for coal fired power plants.

The sensor system is designed so that approximately 12 readings a day may be conducted using a single sensor chamber connected to a fixed point in an alumina refinery. By duplicating the number of sensor chambers more readings may be obtained. It is anticipated that a sample cylinder will be used to sample the alumina refinery stream. This cylinder may be charged within a minute or alternatively could be charge over a half hour or one hour period to provide averaged sampling. This would depend on the requirements of the alumina refinery plant managers. Ideally this would be real-time analysis.

It should be noted that the developed gold sensitive surfaces, although applied to a QCM in the context of this project, may be applied to other sensor platforms. The developed film could be used in resistive gold film sensors or much more sensitive acoustic mass based sensors. For example, the family of Surface Acoustic Wave (SAW) devices would be most suitable for low concentration measurements in the parts per billion (i.e. approximately up to 100 times more sensitive than QCM sensors). In the system of this invention as shown in FIG. 18, the pump is at the front of the process. Additionally a heated sample cylinder may be used, where a dilution ratio may be applied if required.

By using this setup the pressure in the sensor chamber may be controlled at pressures above atmospheric pressure. In a laboratory setup tests are conducted at approximately 23 psi.

Once the sample cylinder is charged with the stream sample, the (diluted) sample is then sent down heated umbilical lines to a heated Mass Flow Controller (MKS MFC 330AH). A 1:4 dilution ratio is preferred. The MFC feeds the gas into the sensor chamber at a controlled rate of 200 sccm. As the VOC, water vapour and mercury concentration is low enough, due to the dilution, the accuracy is improved as the gases/vapours are prevented from condensing out of the gas phase.

It should be noted that a potential negative effect of placing the pump before the sensor chamber may be that the pump could interfere with the integrity of the sample.

An appropriate pump that does not shear the gas molecules may be chosen. The pump head may be heated

### EXAMPLE

### Mercury Sensing

The nanospike and nanoprism structures have high activity and have been observed to have increased response magnitude toward mercury vapour when compared to non-modified surfaces. FIG. 9 shows a typical sensor response towards 5 pulses of mercury vapour between the concentration range of 1.02 and 10.55 mg/m³ at an operating temperature of 89° C. (±3° C.). It can be seen that the nanospike sensor has a large response magnitude up to 180% higher than the non-modified. Similarly FIG. 10 demonstrates that alternative nanostructures formed by the variations of the methods detailed herein can also show comparable sensor performance: a) non-modified, b) poorly formed electroplated surface, c) short nanoprisms, d) nanoprisms and e) an alternative nanospike surface. Both the nanoprisms and nanospikes are shown to have comparable performance.

It should be noted that the most tested nanostructures are the nanospikes. A sensor with nanospike surface has been vigorously tested and has shown good stability over two separate long term tests. The first test totalled 70 days of testing at an operating temperature of 89° C. (±3° C.) over two distinct test periods. The first being a 59 day test (25 days+34 days with ammonia and low level humidity interference using up to 10.4 mg/m³ of H₂O vapour) and a further 11 day test for more interference testing conducted 56 days after the first testing period. During the 56 day non-testing period the sensors were stored at room temperature.

The significance of the results is highlighted in FIG. 9, FIG. 11, FIG. 12 and FIG. 13. Response magnitudes of the nanospikes sensor are show to be up to 180% larger, where a 66% increase in signal-to-noise (S/N) ratio is observed in comparison with non-modified QCM. FIG. 11 shows how there are minimal humidity effects and also low temperature fluctuation effect on the response magnitude of the nanospike sensor. Thus, minor fluctuations in operating temperature will not alter the sensor results significantly. A factorial like testing pattern was used to generate the data shown in FIG. 13. The sensors were exposed to 5 fixed concentrations of mercury (Hg) in dry nitrogen and in the presence of known concentrations of Ammonia (NH₃) and humidity (H₂O). Example response curves from the test sequences can be seen in FIGS. 13. Change in frequency (Δf) and the rate of change (Δf/Δt) were calculated for each test sequence. Although not all possible permeations were undertaken, the tests were designed to acquire a spread of data which represented as many possible combinations with comparable pulses in the restricted time frame. The comparable pulses were used to gather degradation data (i.e. reduction in response magnitude vs. age of sensor) and confirm response repeatability of each sensor. Analysis of the data taken at comparable points during the course of testing revealed that the electro-deposited sensors' response magnitude degraded ˜9% while the non-modified degraded by up to 23.3% over the testing period.

The data is summarised in the following table:

| | Response of | Response of | | Degrada- |
|---|---|---|---|---|
| | Modified QCM | Non-modified QCM | Degrada- | tion % of |
| | to 10.5 mg/m³ | to 10.5 mg/m³ | tion % of | non- |
| Day | of Hg | of Hg | modified | modified |
| 6 | 579.7 Hz | 213.0 Hz | 0.0 | 0.0 |
| 59 | 527.7 Hz | 184.2 Hz | −9.0 | −13.5 |
| 115 | 532.3 Hz | 163.4 Hz | −8.2 | −23.3 |
| 126 | 544.9 Hz | 168.6 Hz | −6.0 | −20.8 |

| | |
|---|---|
| 127+ | Continued testing is required, however it appears that the response |
| | magnitude of both sensors has plateaued. |

The tables below further highlight the significance of the electro-deposited QCM when compared to the non-modified sensor. It is clear that the standard deviation of the sensors appear to be near identical in magnitude, however the larger response magnitude of the electrodeposited sample means that percentage (%) error is at least 1.4 and up to 8 times higher for the non-modified QCM. Coefficient of Variance (CoV) is shown in each case.

Modified by electro-deposition (First long term test):
Δf data

| | Number | | Standard | | | |
|---|---|---|---|---|---|---|
| | of data | Mean | deviation, CoV | Median | Minimum | Maximum |
| Hg Concentration | points | (Hz) | (Hz, [%]) | (Hz) | (Hz) | (Hz) |
| 1.01 | 62 | 211.7 | 13.9 [6.6%] | 217.6 | 177.0 | 229.3 |
| 1.87 | 42 | 281.2 | 13.7 [4.9%] | 282.4 | 254.5 | 306.0 |
| 3.65 | 47 | 360.0 | 11.8 [3.3%] | 358.0 | 333.2 | 385.6 |
| 5.70 | 42 | 452.0 | 14.9 [3.3%] | 450.8 | 414.0 | 487.1 |
| 10.5 | 42 | 534.9 | 23.8 [4.4%] | 536.0 | 496.6 | 584.3 |

Δf/Δt data

| | Number | | Standard | | | |
|---|---|---|---|---|---|---|
| | of data | Mean | deviation, CoV | Median | Minimum | Maximum |
| Hg Concentration | points | (Hz/h) | (Hz/h, [%]) | (Hz/h) | (Hz/h) | (Hz/h) |
| 1.01 | 62 | 857.4 | 25.1 [3.0%] | 857.2 | 805.8 | 915.4 |
| 1.87 | 42 | 1585.7 | 50.1 [3.2%] | 1577.4 | 1516.7 | 1740.9 |
| 3.65 | 47 | 2749.8 | 96.0 [3.5%] | 2745.5 | 2563.8 | 2940.5 |
| 5.70 | 42 | 4438.6 | 220.6 [5.0%] | 4455.6 | 4050.1 | 4893.8 |
| 10.5 | 42 | 6363.1 | 445.8 [7.0%] | 6222.9 | 5614.6 | 7242.1 |

Adsorption:
Minimum CoV Δf = 3.3% Δf/Δt = 3.0%
Maximum CoV Δf = 6.6% Δf/Δt = 7.0%

Non-modified (First long term test):
Δf data

| | Number | | Standard | | | |
|---|---|---|---|---|---|---|
| | of data | Mean | deviation, CoV | Median | Minimum | Maximum |
| Hg Concentration | points | (Hz) | (Hz, [%]) | (Hz) | (Hz) | (Hz) |
| 1.01 | 62 | 72.0 | 10.3 [14%] | 72.6 | 48.3 | 94.2 |
| 1.87 | 42 | 95.7 | 10.4 [11%] | 98.5 | 72.6 | 116.4 |
| 3.65 | 47 | 123.1 | 11.5 [9.3%] | 125 | 99 | 143.6 |
| 5.70 | 42 | 152.3 | 11.4 [7.5%] | 153.8 | 125.1 | 175.7 |
| 10.5 | 42 | 179.2 | 11.0 [6.1%] | 177.4 | 161.3 | 202.9 |

Δf/Δt data

| | Number | | Standard | | | |
|---|---|---|---|---|---|---|
| | of data | Mean | deviation, CoV | Median | Minimum | Maximum |
| Hg Concentration | points | (Hz/h) | (Hz/h, [%]) | (Hz/h) | (Hz/h) | (Hz/h) |
| 1.01 | 62 | 306.2 | 77.2 [25%] | 282.1 | 223.7 | 458.8 |
| 1.87 | 42 | 523.6 | 130.5 [25%] | 513.5 | 346.8 | 773.5 |
| 3.65 | 47 | 889.8 | 246.9 [28%] | 895.6 | 559.7 | 1274.1 |
| 5.70 | 42 | 1304.9 | 341.6 [26%] | 1330.6 | 855.8 | 1909.1 |
| 10.5 | 42 | 1849.7 | 474.5 [25%] | 1809.4 | 1257.5 | 2730.9 |

Adsorption:
Minimum CoV Δf = 6.1% Δf/Δt = 25%
Maximum CoV Δf = 14% Δf/Δt = 28%

The second test totalled 95 days of testing at an operating temperature of 102° C. over a single continuous testing period. Using pattern sequences like those shown in FIGS. 13 and 14 the sensors were exposed to a wider range of interfering gases during the adsorption phase of the sensing pulse. Interfering gas species included Ammonia, Dimethyl disulphide, Ethyl Mercaptan, Methyl Ethyl Keytone, Acetaldehyde and a high level humidity interference using up to 23 g/m³ of H₂O vapour. The significance of the results is highlighted in FIG. 14 through to FIG. 17. FIG. 14 clearly shows that the electrodeposited nanospike sensor has a superior signal-to-noise ratio and significantly larger response magnitude.

FIGS. 15 and 16 summaries the performance of both the electrodeposited nanospikes and non-modified QCMs for the adsorption and desorption phase of the sensing event, respectively, which were collected during the 95 day testing period. Box plots with 25% and 75% quartiles were chosen to represent all data points collected for each group, where the whiskers represent the standard deviation (SD) and the asterisks represent the minimum and maximum values obtained for each test type. The sample set size, n, indicates the number of data points represented by each box. It is clear from the data spread that the electrodeposited QCM significantly outperforms the non-modified sensor. The tables below further highlight the significance of the electrodeposited QCM when compared to the non-modified sensor.

Modified by electrodeposition (Second long term test):
Δf data

| | Number | | | | Standard | | | | | | | |
|---|---|---|---|---|---|---|---|---|---|---|---|---|
| | of data | | Mean | | deviation, CoV | | Median | | Minimum | | Maximum | |
| Hg | points | | (Hz) | | (Hz, [%]) | | (Hz) | | (Hz) | | (Hz) | |
| (mg/m³) | ads | des | ads | des | ads | des | ads | des | ads | des | ads | des |
| 1.01 | 210 | 203 | 108.0 | 117.6 | 14.9 [14%] | 9.7 [8.2%] | 111.0 | 114.6 | 53.81 | 103.5 | 142.3 | 148.8 |
| 1.87 | 182 | 175 | 161.7 | 165.5 | 11.7 [7.2%] | 9.3 [5.6%] | 162.5 | 163.7 | 118.0 | 148.8 | 198.8 | 196.2 |
| 3.65 | 182 | 175 | 228.1 | 226.5 | 14.9 [6.5%] | 8.5 [3.7%] | 226.1 | 225.6 | 198.5 | 207.3 | 282.9 | 255.3 |
| 5.70 | 182 | 175 | 304.4 | 297.8 | 19.3 [6.3%] | 8.7 [2.9%] | 300.1 | 297.7 | 271.7 | 274.7 | 374.9 | 318.1 |
| 10.5 | 182 | 175 | 390.0 | 379.4 | 23.7 [6.1%] | 8.2 [2.2%] | 383.6 | 380.7 | 338.6 | 358.9 | 470.0 | 394.9 |

Δf/Δt data

| | Number | | | | Standard | | | | | | | |
|---|---|---|---|---|---|---|---|---|---|---|---|---|
| | of data | | Mean | | deviation, CoV | | Median | | Minimum | | Maximum | |
| Hg | points | | (Hz/h) | | (Hz/h, [%]) | | (Hz/h) | | (Hz/h) | | (Hz/h) | |
| (mg/m³) | ads | des | ads | des | ads | des | ads | des | ads | des | ads | Des |
| 1.01 | 210 | 210 | 226.2 | 192.9 | 25.3 [11%] | 19.2 [10%] | 229.9 | 188.0 | 151.1 | 162.1 | 316.8 | 257.5 |
| 1.87 | 182 | 182 | 399.3 | 301.1 | 34.7 [8.7%] | 22.4 [7.4%] | 402.4 | 297.0 | 314.6 | 257.1 | 503.5 | 393.8 |
| 3.65 | 182 | 182 | 629.6 | 445.0 | 60.1 [9.5%] | 27.2 [6.1%] | 629.9 | 445.4 | 472.2 | 359.1 | 805.6 | 522.5 |
| 5.70 | 182 | 182 | 866.8 | 625.5 | 77.7 [9.0%] | 39.6 [6.3%] | 864.5 | 627.0 | 608.0 | 520.4 | 1078 | 801.8 |
| 10.5 | 182 | 182 | 1078 | 836.1 | 77.5 [0.7%] | 40.3 [4.8%] | 1072 | 837.1 | 874.4 | 728.6 | 1314 | 1067 |

Adsorption:
Minimum CoV Δf = 6.1% Δf/Δt = 0.7%
Maximum CoV Δf = 14% Δf/Δt = 11%
Desorption:
Minimum CoV Δf = 2.2% Δf/Δt = 4.8%
Maximum CoV Δf = 8.2% Δf/Δt = 10%

Non-modified (Second long term test):
Δf data

| | Number | | | | Standard | | | | | | | |
|---|---|---|---|---|---|---|---|---|---|---|---|---|
| | of data | | Mean | | deviation, CoV | | Median | | Minimum | | Maximum | |
| Hg | points | | (Hz) | | (Hz, [%]) | | (Hz) | | (Hz) | | (Hz) | |
| (mg/m³) | ads | des | ads | des | ads | des | ads | des | ads | des | ads | des |
| 1.01 | 210 | 203 | 61.0 | 63.1 | 4.5 [7.4%] | 5.5 [8.7%] | 60.9 | 62.1 | 51.7 | 50.7 | 79.6 | 79.2 |
| 1.87 | 182 | 175 | 84.5 | 85.4 | 5.2 [6.1%] | 5.7 [6.7%] | 84.2 | 84.9 | 74.1 | 73.3 | 97.6 | 98.1 |
| 3.65 | 182 | 175 | 112.9 | 112.6 | 5.4 [4.8%] | 5.4 [4.8%] | 112.6 | 112.6 | 101.3 | 98.1 | 130.8 | 128.7 |
| 5.70 | 182 | 175 | 143.8 | 142.2 | 6.0 [4.2%] | 5.3 [3.7%] | 142.9 | 141.6 | 132.0 | 130.4 | 161.5 | 156.2 |
| 10.5 | 182 | 175 | 173.6 | 171.1 | 6.2 [3.6%] | 4.9 [2.9%] | 173.0 | 170.3 | 160.8 | 160.9 | 188.3 | 188.3 |

Δf/Δt data

| | Number | | | | Standard | | | | | | | |
|---|---|---|---|---|---|---|---|---|---|---|---|---|
| | of data | | Mean | | deviation, CoV | | Median | | Minimum | | Maximum | |
| Hg | points | | (Hz/h) | | (Hz/h, [%]) | | (Hz/h) | | (Hz/h) | | (Hz/h) | |
| (mg/m³) | ads | des | ads | des | ads | Des | ads | des | ads | des | ads | des |
| 1.01 | 210 | 210 | 147.2 | 122.7 | 21.0 [14%] | 14.3 [12%] | 147.8 | 120.4 | 91.0 | 92.8 | 224.8 | 167.9 |
| 1.87 | 182 | 182 | 202.8 | 177.0 | 32.6 [16%] | 17.8 [10%] | 202.4 | 174.6 | 119.5 | 136.2 | 278.2 | 245.2 |
| 3.65 | 182 | 182 | 287.7 | 247.4 | 39.2 [14%] | 21.7 [8.8%] | 289.9 | 245.5 | 174.4 | 174.0 | 396.5 | 319.0 |
| 5.70 | 182 | 182 | 360.2 | 324.5 | 40.2 [11%] | 26.0 [8.0%] | 359.6 | 319.5 | 215.2 | 275.9 | 490.6 | 435.1 |
| 10.5 | 182 | 182 | 412.4 | 396.4 | 36.1 [8.8%] | 26.9 [6.8%] | 410.1 | 391.8 | 223.5 | 341.4 | 507.1 | 518.1 |

Adsorption:
Minimum CoV Δf = 3.6% Δf/Δt = 8.8%
Maximum CoV Δf = 7.4% Δf/Δt = 14%
Desorption:
Minimum CoV Δf = 2.9% Δf/Δt = 6.8%
Maximum CoV Δf = 8.7% Δf/Δt = 16%

In comparison to the non-modified, the electrodeposited nanospike sensor has the following advantages:
has better temperature stability,
is estimated to have around 3 times longer usable lifetime,
is stable under the tested humidity and chemicals/VOC interference concentrations,
has a better S/N at elevated operating temperatures.

Therefore the data above strongly suggests that the electrodeposited mercury sensor with the nanospike structures is extremely well suited and a huge step forward towards producing an on-line elemental mercury sensor for refinery streams. It is capable of dealing with fluctuating operating temperature, high level of humidity and interference from many chemicals/VOCs commonly found in refinery gas streams.

From the above it can be seen that this invention provides a unique sensing surface that provides potential for improved sensing of mercury vapour in an industrial environment.

Those skilled in the art will realise that this invention may be implemented in embodiments other than those described without departing from the core teachings of this invention.

1. A method of forming gold nanostructures on a gold substrate which includes the steps of electrodepositing gold onto a gold working electrode from a solution of hydrogen or alkali metal tetrahaloaureate (III) and growth directional additive at an electro-deposition temperature between 20 and 40° C. and a deposition time of at least 15 seconds.

2. A method as claimed in claim 1 in which the growth directional additive is selected from various lead salts, halides, saccharin, Nafion, CTAB, SDS, Triton, and cysteine.

3. A method as claimed in claim 1 in which the deposition solution contains 2.718 g/l of hydrogen tetrachloroaurate(III) hydrate with 0.1 to 0.5 g/l of lead acetate.

4. A method as claimed in any preceding claim in which the deposited nano structures are heated for a prolonged period of time at a temperature above 150° C.

5. A method as claimed in any preceding claim in which a constant current between 0.1 mA and 5 mA is used.

6. A method as claimed in any one of claims 1-4 in which a constant potential difference between 0.2V and 3V is used.

7. A chemical and biological sensor in which the sensor surface is a metallised substrate and gold nanostructures with controlled crystallographic facets are strongly adhered to the substrate using a deposition method as claimed in claim 1.

8. A mercury vapour sensor in which the sensor surface is a metallised substrate to which gold nanostructures with controlled crystallographic facets are strongly adhered to the substrate with interstitial spacing.

9. A mercury vapour sensor as claimed in claim 8 which includes hierarchical nanostructures with two-tier roughness in the form of secondary nodes on the primary structures.

10. A mercury vapour sensor system using the extractive dilution technique in which samples are collected, diluted and passed through a sensor chamber containing a sensor as claimed in claim 8 or 9.

11. A mercury vapour sensor system as claimed in claim 10 in which the pressure in the sensor chamber is above atmospheric pressure.
